(19) United States
(12) Patent Application Publication
(10) Pub. No.: US 2006/0057238 A1
(43) Pub. Date: Mar. 16, 2006

(54) Extract of buds of cryptomeria japonica d. don

(75) Inventors: Frederic Demarne, Marseille (FR); Anne-Emmanuelle Guise-Ganz, Lyon (FR)

Correspondence Address: HESLIN ROTHENBERG FARLEY & MESITI PC, 5 COLUMBIA CIRCLE, ALBANY, NY, 12203, US

(73) Assignee: Gattefosse Sas, Saint Priest (FR)

(21) Appl. No.: 10/542,323

(22) PCT Filed: Dec. 29, 2003
(86) PCT No.: PCT/FR03/50214

§ 371 Date: Jul. 13, 2005

(30) Foreign Application Priority Data
Jan. 27, 2003 (FR) FR03/00839
Mar. 14, 2003 (FR) FR03/03152

(51) Int. Cl. A61K 36/185

(52) U.S. Cl. 424/777

(57) ABSTRACT

The invention relates to extract of buds of <i>Cryptomeria japonica D. Don</I> which may be obtained by means of a first solid/liquid extraction stage, followed by a second solid/liquid separation stage and finally a third stage of recovery of the liquid phase.

The invention relates to an extract of Cryptomeria japonica buds. It also concerns a cosmetic composition containing said extract. Lastly, it reports various uses in the cosmetic field, by topical application, of the extract and therefore of the composition of the invention.

Originally from China and Japan, Cryptomeria japonica D. Don is an insular species, the sole representative of its genus in the Taxodiaceae family. This fast-growing resinous tree is very robust and thrives in cool, moist, slightly calcareous soil in Europe, but notably in Japan, where, along with the Hinoki False Cypress (Chamaecyparis obtusa) and the Japanese red pine (Pinus densiflora), it covers nearly 40% of the forest area.

To the Applicant's knowledge, only the wood, bark and leaves of Cryptomeria japonica have been used.

In a first application, the wood is used in construction as formwork lumber, for making paneling or for country furniture. A derivative of wood, wood charcoal, has already been described in document JP 2001 302444, in a cosmetic application, for improving the humectant properties of skin or hair. In practice, the cosmetic composition comes in liquid form and is obtained by elution of mineral water on a wood charcoal support.

The bark of Cryptomeria japonica is used as an antimicrobial agent to control pathogenic microorganisms in plants. Thus, document JP 011292727 describes an antimicrobial agent obtained by extraction from Cryptomeria japonica bark using a non-polar organic solvent.

The leaves of Cryptomeria japonica had a first application in the medical field. Thus, document JP 011228433 describes an antibacterial agent, notably directed against Escherichia coli or Legionella, combining a plant extract comprising 35 types of plants, including Cryptomeria japonica, and an organic molecule containing a tropolone nucleus, in the presence of an emulsifying agent and an organic acid. This document does not indicate which part of Cryptomeria japonica is used. Document JP 2001 000141 describes an extract of Cryptomeria japonica leaves used to prevent allergic illnesses. For the same application, document JP 01061415 describes a composition based on extracts of rhizomes, roots or leaves of various plants, notably leaves of Cryptomeria japonica.

In the cosmetic field, document JP 2001 03719 describes a topical composition that improves the appearance of the skin, combining lemon extract, aloe and Cryptomeria japonica leaves.

In other terms, no document describes the idea of using the buds of Cryptomeria japonica rather than the wood, leaves or bark. And yet, the Applicant observed that, surprisingly, extracts of Cryptomeria japonica buds have interesting properties when they are applied to the skin.

In other words, and according to a first aspect, the invention relates to an extract of Cryptomeria japonica buds which may be obtained by a first solid/liquid extraction step, followed by a second solid/liquid separation step, then a third liquid phase retrieval step.

According to a first feature, the solid/liquid extraction may be performed using various techniques that are well known to those skilled in the art, such as maceration, remaceration, digestion, dynamic maceration, fluid bed extraction, microwave-assisted extraction, ultrasound-assisted extraction, countercurrent extraction, percolation, repercolation, leaching, low-pressure extraction, diacolation, supercritical fluid extraction or solid-liquid extraction with continuous reflow (soxhlet). In an advantageous embodiment, extraction is performed by hot dynamic maceration.

According to a further feature, solid/liquid extraction is performed using buds that are fresh, dried, fresh treated with microwave frequencies, or fresh treated with microwave frequencies and then dried; the buds may be also presented whole, crushed, ground or cryoground.

Moreover, the extraction solvent corresponding to the liquid phase is an organic solvent which may be used topically in cosmetic applications. The extraction solvent is chosen from among the group including water, alcohols (ethanol, methanol, etc.), glycols (such as propylene glycol, butylene glycol, glycerin, etc.), alone or in mixtures.

In practice, the bud/solvent ratio in the extraction step is between 1/99 and 80/20 (by weight). Likewise, extraction is performed at a temperature between 3° C. and 100° C., preferably between 20° C. and 60° C., for a few minutes to several days, depending on the extraction method used.

To optimize the extraction of active compounds while protecting said compounds from oxidation by oxygen in the air, the solid/liquid extraction step may be performed while stirring and in nitrogen atmosphere.

According to the invention, solid/liquid extraction is followed by a solid/liquid separation step whose objective is to retrieve the liquid phase containing the active material. This separation may be performed by any technique known to those skilled in the art, notably draining, pressing, spin drying, centrifugation or filtration.

In an advantageous embodiment, the liquid/solid separation step is followed by at least one clarification step. This clarification step may be performed by plate filtration, membrane filtration, tangential filtration or by centrifugation.

According to another embodiment, the liquid/solid separation step is followed by a concentration step, which provides a concentrated liquid form. In practice, the concentration step is performed by vacuum evaporation or reverse osmosis. Of course, the concentration step may be performed directly after the separation or clarification step.

After the solid/liquid separation step, in another embodiment, the extract obtained is fractioned, enriched or purified by various techniques, such as membrane filtration, liquid/liquid extraction or preparative chromatography.

Lastly, for sterile or non-sterile packaging, the clarification and/or concentration steps may be followed by a sterilizing 0.22-μm filtration step.

As stated above, at the end of the separation step, an extract is retrieved in liquid form. To obtain a liquid extract that is stable over time in terms of bacterial contamination, physicochemical stability and color, if a non-sterile packaging is used, at least one preservative agent (Phenonip®, for example) is added in the liquid phase if necessary before the sterilizing filtration step, in a concentration between 1 and 10 g/l, along with an antioxidant agent (for example, organic acids: ascorbic acid, citric acid, etc.) in a concentration between 0.5 and 10 g/l of the total volume of the liquid phase.

To obtain a dry extract, the extract obtained at the end of the liquid/solid separation step, or where applicable the clarification and/or concentration step, is dried with or without a preservation agent and with or without a texturizing agent (such as starch, maltodextrins, corn syrups, etc.), by lyophilization, atomization or vacuum evaporation.

According to a further feature, when the extract is in liquid form, it has a dry matter content between 1 and 100 g/kg. When it is in dry form, it has a dry matter content between 10 and 1000 g/kg.

The extract may be used in the cosmetic field, notably when applied topically. Thus, the Applicant has observed that the extract in the invention:
stimulated the synthesis of essential components of the extracellular matrix through the dermis cells;
had cytoprotective activity on the skin;
stimulated epidermal cell metabolism.

In other words and according to a further aspect of the invention, the aforementioned extract may be used in these applications.

Stimulation of epidermal cell metabolism was demonstrated by the Applicant, who has shown that the extract in the invention has an effect on the respiration of epidermal cells, notably keratinocytes. Moreover, it appears that this response does not correspond to a poison effect, which could be caused by the extract, but to a veritable energizing effect. This stimulation of the cell metabolism thus makes it possible to achieve homeostasis, i.e. a balance between proliferation/differentiation of cells within the epidermis.

This property of the extract of the invention means that the composition in the invention may be used, in topical application, as an age-defying, moisturizing, normalizing and stimulating agent for the complexion's radiance and therefore a cosmetic treatment method consisting in applying said composition to the skin.

According to a further aspect, the invention relates to a cosmetic composition containing an extract of Cryptomeria japonica buds, notably an extract obtained using the aforementioned method.

In practice, the extract represents between 0.1% and 10% of the composition by weight, preferably between 0.3% and 3%.

The composition according to the invention may be presented in all the galenic forms normally used for topical application on the skin or hair, notably in the form of an aqueous solution, an oil-water, water-oil or multiple emulsion, a silicon emulsion, a microemulsion or nanoemulsion or an aqueous gel.

This composition may be more or less fluid and may take on the appearance, among others, of a white or colored cream, a pomade, a milk, a lotion, a serum or a gel.

The composition of the invention may contain adjuvants commonly used in the cosmetics and dermatology fields, such as fats, emulsifiers and co-emulsifiers, hydrophilic or lipophilic gelling agents, active hydrophilic or lipophilic ingredients, preservatives, antioxidants, solvents, scents, fillers, hydrophilic or lipophilic filters, colorants, neutralizers, pro-penetrating agents and polymers.

The quantities of the various adjuvants are those conventionally used in the fields in question and, for example, from 0.01% to 30% of the total weight of the composition. These adjuvants, depending on their nature, may be included in the oil phase or in the aqueous phase.

The fats that may be used in the invention include mineral oils, oils of animal origin (lanolin), synthetic oils (isopropyl myristate, octyldodecyl, isostearyl isostearate, decyl oleate, isopropyl palmitate), silicon oils (cyclomethicone, dimethicone) and fluorinated oils. The following may be used as fats: fatty alcohols, fatty acids, waxes and gums, notably silicon gums and elastomers.

The emulsifiers and co-emulsifiers that may be used in the invention include, for example, polyglycerol and fatty acid esters, sucrose and fatty acid esters, sorbitan- and fatty acid esters, fatty acid and oxyethylene sorbitan esters, fatty alcohol and PEG esters, glycerol and fatty acid esters, alkyl sulfates, alkyl ether sulfates, alkyl phosphates, alkyl polyglucosides and dimethicone copolyols.

The hydrophilic gelling agents that may be used in the invention notably include carboxyvinyl polymers (carbomer), acrylic copolymers such as acrylate/alkylacrylate copolymers, polyacrylamides, polysaccharides such as xanthan gum, guar gum, natural gums such as cellulose gum and derivatives, clays and copolymers of 2-acrylamido-2-methylpropane acid.

The lipophilic gelling agents that may be used in the invention include modified clays such as bentones, metallic salts of fatty acids, hydrophobic silica and ethylcellulose.

The cosmetic composition may also contain active ingredients. These active ingredients may notably include depigmentation agents, emollients, moisturizers, anti-seborrhea agents, anti-acne agents, keratolytic and/or scaling agents, anti-wrinkle and firming agents, draining agents, anti-irritant agents, soothing agents, slimming agents such as xanthic bases (caffeine), vitamins and their mixtures, and matting agents.

In case of incompatibility among them or with the Cryptomeria japonica D. Don extract, the active ingredients indicated above and/or the Cryptomeria japonica D. Don extract may be encapsulated in spherules, notably ionic or non-ionic vesicles and/or nanoparticles (nanocapsules and/or nanospheres), to isolate them from each other in the composition.

The preservatives that may be used in the invention include benzoic acid, its salts and its esters; sorbic acid and its salts; parabens, their salts and esters; triclosan; imidazolidinyl urea; phenoxyethanol;
DMDM hydantoin; diazolidinyl urea and chlorphenesin.

The antioxidants that may be used in the invention include chelating agents such as EDTA and its salts.

The solvents that may be used in the invention include water, ethanol, glycerin, propylene glycol, butylene glycol and sorbitol.

The fillers that may be used in the invention include talc, kaolin, mica, serecite, magnesium carbonate, aluminum silicate, magnesium silicate and organic powders such as nylon.

The filters that may be used in the invention include conventionally used UVA and UVB filters such as benzophenone-3, butyl methoxydibenzoyl methane, octocrylene, octyl methoxycinnamate, 4-methylbenzylidene camphor, octyl salicylate, terephthalylidene dicamphor sulfanic acid and drometrizole trisiloxane. Others can be mentioned such as TiO₂ and ZnO physical filters in their micrometric and nanometric forms.

The colorants that may be used in the invention include lipophilic colorants, hydrophilic colorants, pigments and mother-of-pearl conventionally used in cosmetic or dermatological compositions, and their mixtures.

The neutralizers that may be used in the invention include soda, triethanolamine, aminomethyl propanol and potassium hydroxide.

The pro-penetrating agents that may be used in the invention include alcohols and glycols (ethanol, propylene glycol), ethoxydiglycol, fatty alcohols and acids (oleic acid), fatty acid esters and dimethyl isosorbide.

The composition according to the invention may be used as a care product (for example as a slimming product), as a cleansing product and/or as a skin makeup product, as a sunscreen product or as a hair care product, for example as a shampoo or conditioner.

The invention and the advantages it provides will become more apparent with the description of the following exemplary embodiments.

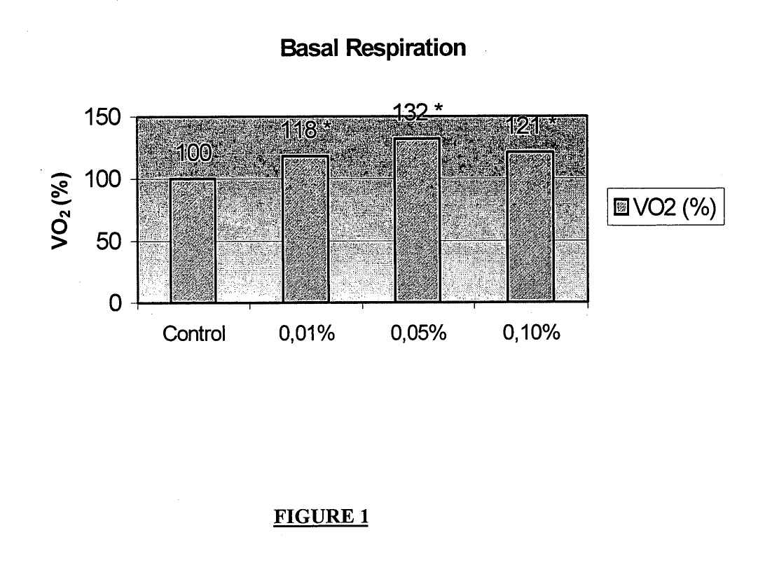

FIG. 1 represents the effect of the extract of the invention on the basal respiration of human keratinocytes.

### EXAMPLE 1

### Production of an Extract of Cryptomeria japonica Buds

Place 473.7 g butylene glycol and 426.3 g purified water in the same beaker;
Heat the solvent to 40° C., stirring constantly;
Weigh out 100 g of frozen Cryptomeria japonica buds;
Grind the buds for a few seconds with a cutting mill;
Add the ground buds to the water/butylene glycol mixture;
Allow to extract for approximately 8 hours at 40° C., stirring constantly;
Remove the buds by passing through a nylon sheet (100 μm);
Clarify the extract using paper filters with decreasing porosity.

### EXAMPLE 2

### Cosmetic Composition

Face Cream

| | | |
|---|---|---|
| PEG-8 beeswax | O/W emulsifier | 5.00 |
| Stearic acid | Thickener | 1.50 |
| Cyclomethicone | Emollient | 10.00 |
| Phenyl trimethicone | Emollient | 5.00 |
| Phenoxyethanol and methylparaben and | Preservative | 0.50 |
| butylparaben and ethylparaben and | | |
| propylparaben | | |
| Acrylates/Steareth-20 methacrylate | Gelling agent | 1.00 |
| copolymer | | |
| Sodium hydroxide (10% sol.) | Neutralizing agent | 0.40 |
| Dimethicone and dimethiconol | Texturizing agents | 4.00 |
| Extract of Cryptomeria japonica buds | | 3.00 |
| Water | | qs 100 |

Slimming Body Gel

| Composition | % w/w |
|---|---|
| Carbomer | 0.2 |
| Butylene glycol | 12.0 |
| Phenoxyethanol, methylparaben, butylparaben, | 1.0 |
| ethylparaben, propylparaben | |
| Sodium hydroxide (10% sol.) | 0.4 |
| Alcohol | 20.0 |
| Ethoxydiglycol | 4.0 |
| Liquid extract of Cryptomeria japonica | 5.0 |
| Glyceryl polymethacrylate and propylene glycol | 10.0 |
| Water | qs 100.0 |

Slimming Body Milk

| Composition | % w/w |
|---|---|
| PEG-6 stearate and ceteth-20 and steareth-20 | 8.0 |
| Propylene Glycol Dipelargonate | 10.0 |
| Stearic acid | 1.0 |
| Hydrogenated castor oil | 1.0 |
| Apricot stone oil | 3.0 |
| Dimethicone | 2.0 |
| Tocopheryl acetate | 0.5 |
| Polydecene | 3.0 |
| Cyclomethicone | 3.0 |
| Phenoxyethanol, methylparaben, butylparaben, | 1.0 |
| ethylparaben and propylparaben | |
| Carbomer | 0.15 |
| Xanthan gum | 0.3 |
| Ethanol | 5.0 |
| Glycerin | 3.0 |
| Sodium hydroxide (10% sol.) | 0.3 |
| Liquid extract of Cryptomeria japonica | 3.0 |
| Ascorbic acid | 0.05 |
| Scent | 0.4 |
| Water | qs 100.0 |

O/W Emulsion

| Composition | Quantity (%) |
|---|---|
| Phenoxyethanol, Methylparaben, Butylparaben, | 1 |
| Ethylparaben, Propylparaben | |
| Carbomer | 0.4 |
| Glycerin | 3 |
| Xanthan gum | 0.1 |
| Polysorbate-60 | 0.9 |
| Glyceryl Stearate, PEG-100 Stearate | 2.1 |
| Cetyl Alcohol | 2.6 |
| Paraffin oil | 7.5 |
| Isopropyl Myristate | 7.5 |
| Ethoxydiglycol | 5 |
| Dry extract of Cryptomeria japonica | 1 |
| Scent | 0.2 |
| Triethanolamine | 0.3 |
| Water | qs 100 |

W/O Emulsion

| Composition | Quantity (%) |
|---|---|
| Glycerin | 3 |
| Propylene Glycol, Diazolidinyl Urea, Methylparaben, | 1 |
| Propylparaben | |
| Magnesium Sulfate | 0.7 |
| Cetyl Dimethicone Copolyol | 2.5 |
| Isohexadecane | 5 |
| Caprylic/Capric Triglyceride | 5 |
| Dimethicone | 5 |
| Alcohol | 5 |
| Dry extract of Cryptomeria japonica | 2 |
| Scent | 0.1 |
| Water | qs 100 |

Microemulsion

| Composition | Quantity (%) |
|---|---|
| PEG-8 Caprylic/Capric Glycerides | 13.33 |
| Polyglyceryl-6 Dioleate | 8.67 |
| Isostearyl Isostearate | 4 |
| Cyclomethicone | 2.3 |
| Diisopropyl Adipate | 1.6 |
| Octyldodecanol | 2 |
| PPG-5 Ceteth-20 | 2 |
| Phenoxyethanol, Methylparaben, Butylparaben, | 0.4 |
| Ethylparaben, Propylparaben | |
| Ethoxydiglycol | 2 |
| Dry extract of Cryptomeria japonica | 1 |
| Water | qs 100 |

W/O/W Multiple Emulsion

| Composition | Quantity (%) |
|---|---|
| PEG-30 Dipolyhydroxystearate | 2.4 |
| Isohexadecane | 9 |
| PPG-15 Stearyl Ether | 4.5 |
| Caprylic/Capric Triglyceride | 4.5 |
| Magnesium Sulfate | 0.82 |
| Propylene Glycol, Diazolidinyl Urea, Methylparaben, | 1.2 |
| Propylparaben | |
| Dry extract of Cryptomeria japonica | 2 |
| Poloxamer 407 | 2 |
| Glycerin | 3 |
| Xanthan gum | 0.7 |
| Scent | 0.2 |
| Water | qs 100 |

Sunscreen

| Composition | Quantity (%) |
|---|---|
| DEA Cetyl Phosphate | 2 |
| Glyceryl Stearate, PEG-100 Stearate | 4 |
| Beeswax | 2 |
| Octyl Methoxycinnamate | 7 |
| Butyl Methoxydibenzoylmethane | 2 |
| Benzophenone-3 | 1 |
| Titanium Dioxide | 3 |
| C12/C15 Alkyl Benzoate | 3 |
| Cyclomethicone | 2 |
| Tocopheryl Acetate | 0.5 |
| EDTA | 0.1 |
| Acrylates/C10-30 Alkyl Acrylates Crosspolymer | 0.2 |
| Xanthan gum | 0.3 |
| Phenoxyethanol, Methylparaben, Ethylparaben, | 1 |
| Propylparaben, Isobutylparaben | |
| Butylene Glycol | 3 |
| Dry extract of Cryptomeria japonica | 1 |
| Sodium hydroxide (10% sol.) | 0.4 |
| Scent | 0.3 |
| Water | qs 100 |

Makeup Foundation

| Composition | Quantity (%) |
|---|---|
| Glyceryl Stearate, Propylene Glycol Stearate, Glyceryl | 5 |
| Isostearate, Propylene Glycol Isotearate, Oleth-25, | |
| Ceteth-25 | |
| Glyceryl Dibehenate, Tribehenin, Glyceryl Behenate | 1 |
| Ethoxydiglycol Oleate | 7.5 |
| Isostearyl isostearate | 5 |
| Cetearyl Alcohol | 2 |
| Dimethicone | 5 |
| Tocopheryl acetate | 0.5 |
| Phenoxyethanol, Methylparaben, Ethylparaben, | 0.6 |
| Propylparaben, Isobutylparaben | |
| Xanthan gum | 0.4 |
| Microcrystalline Cellulose, Cellulose Gum | 1.5 |
| Titanium Dioxide | 6.6 |
| Iron Oxides (Yellow pigment) | 1.55 |
| Iron Oxides (Red pigment) | 0.43 |
| Iron Oxides (Black pigment) | 0.11 |
| Ethoxydiglycol Oleate | 2.5 |
| Dimethicone, Dimethiconol | 3 |
| Alcohol | 5 |
| Dry extract of Cryptomeria japonica | 2 |
| Water | qs 100 |

Shampoo

| Composition | Quantity (%) |
|---|---|
| Acrylates Copolymer | 1.5 |
| Sodium Laurel Sulfate | 5 |
| Sodium Laureth Sulfate | 4 |
| Cocamidopropyl Betaine | 1.5 |
| Polyquaternium-10 | 0.25 |
| DMDM Hydantoin | 0.3 |
| Sodium Hydroxide (20% solution) | 1.3 |
| Citric Acid (50% solution) | 0.7 |
| Dry extract of Cryptomeria japonica | 0.5 |
| Scent | 0.5 |
| Sodium chloride | 0.5 |
| Water | qs 100 |

### EXAMPLE 3

### Effects of the Extract on the Stimulation of Cell Metabolism

The purpose of the study was to assess the effect of the extract of the invention on epidermal cell metabolism through cell respiration.

The extract is obtained under the same conditions as in example 1. For the study, the extract is placed in a solution in a respiratory buffer at concentrations of 0.01%, 0.05% and 0.1% (v/v).

This activity on cell respiration was assessed by measuring the speed of oxygen consumption (VO₂) by HaCaT human keratinocytes placed in the following experimental conditions:
On normal (non-permeabilized) cells in suspension in a buffer rich in respiratory substrates to observe a modulation of the cell respiration considered overall.
After permeabilization of the cells through partial lysis of the cytoplasmic membrane, conditions which eliminate problems of transport and distribution of the substance to the mitochondria, thus making it possible to observe a modulation in respiration by direct action of the substance on the mitochondria.
After adding a decoupling agent onto the permeabilized cells, which induces maximum mitochondrial respiration. This study is performed with the sole purpose of assessing whether the positive effect of a substance on mitochondrial respiration comes from a decoupling effect or not.

Under the experimental conditions adopted, this study demonstrated that:
The extract is able to stimulate basal respiration in HaCaT keratinocytes. A 32% increase in the apparent speeds of O₂ consumption was observed during incubation of whole (non-permeabilized) cells with the active ingredient at 0.05% (see FIG. 1).
The extract does not modify mitochondrial respiration. No significant modification of the apparent speeds of O₂ consumption was observed during incubation of permeabilized cells with concentrations of the active ingredient between 0.01% and 0.1% (v/v).
The extract does not modify the apparent speeds of O₂ consumption in permeabilized cells in the presence of a decoupling agent (DNP).

The totality of these results allows us to conclude that there is a stimulating effect on cell respiration without a mitochondrial decoupling effect. The stimulation observed in cell respiration was not due to a direct effect of the substance on the mitochondrial respiratory chain. This active ingredient may act upstream from the mitochondria, at the level of glycolysis, glucose transport or as a respiratory substrate.

1. An extract of Cryptomeria japonica D. Don buds obtained by a first solid/liquid extraction step, followed by a second solid/liquid separation step, then a third liquid phase retrieval step.

2. An extract as claimed in claim 1, wherein the solid/liquid extraction step is performed by maceration, remaceration, digestion, dynamic maceration, fluid bed extraction, microwave-assisted extraction, ultrasound-assisted extraction, countercurrent extraction, percolation, repercolation, leaching, low-pressure extraction, diacolation, supercritical fluid extraction or solid-liquid extraction with continuous reflow (soxhlet).

3. An extract as claimed in claim 1, wherein a bud/solvent ratio in the solid/liquid extraction step is between 1/99 and 80/20 by weight.

4. An extract as claimed in claim 1, wherein the solid/liquid extraction step is performed at a temperature between 3° C. and 100° C., preferably between 20° C. and 60° C.

5. An extract as claimed in claim 1, wherein the solid/liquid separation step is performed by draining, pressing, spin drying, centrifugation or filtration.

6. An extract as claimed in claim 1, wherein the solid/liquid separation step is followed by at least one clarification step.

7. An extract as claimed in claim 1, wherein the solid/liquid separation step is followed by a concentration step.

8. An extract as claimed in claim 6, wherein the at least one clarification step is followed by a sterilizing 0.22-μm filtration step.

9. An extract as claimed in claim 1, wherein the extract is dried after the solid/liquid separation step by lyophilization, atomization or vacuum evaporation.

10. An extract as claimed in claim 1, wherein when the extract is in liquid form, it has a dry matter content between 1 and 100 g/kg.

11. An extract as claimed in claim 1, wherein when the extract is in dry form, it has a dry matter content between 10 and 1000 g/kg.

12. A method for stimulating synthesis of essential components of the extracellular matrix through dermis cells comprising applying an extract as claimed in claim 1 to said dermis cells.

13. A method for cytoprotection of the skin comprising applying an extract as claimed in claim 1 to said skin.

14. A method for stimulating epidermal cell metabolism comprising applying an extract as claimed in claim 1 to said epidermal cells.

15. A cosmetic composition containing an extract of Cryptomeria japonica buds as claimed in claim 1.

16. A composition as claimed in claim 15, wherein the extract represents between 0.1% and 10% of the composition by weight, preferably between 0.3% and 3%.

17. A method for moisturizing, normalizing and stimulating the complexion's radiance comprising topically applying the composition of claim 15.

18. An extract as claimed in claim 7, wherein the concentration step is followed by a sterilizing 0.22-μm filtration step.

19. An extract according to claim 1 obtained by:
(1) extracting Cryptomeria japonica D. Don buds with water, alcohol or a polyol to provide a solid phase and a liquid phase;
(2) separating said solid phase and liquid phase; and
(3) clarifying said liquid phase.

20. A process for preparing an extract of Cryptomeria japonica D. Don buds, comprising a first solid/liquid extraction step, followed by a second solid/liquid separation step, followed by a third liquid phase retrieval step.

21. The process as claimed in claim 20, wherein:
the first step comprises extracting Cryptomeria japonica D. Don buds with water, alcohol or a polyol to provide a solid phase and a liquid phase;
the second step comprises separating said solid phase from said liquid phase; and
the third step comprises clarifying said liquid phase.
